(19) United States
(12) Plant Patent Application Publication
(10) Pub. No.: US 2011/0145959 P1
(43) Pub. Date: Jun. 16, 2011

(54) Agastache plant named 'Summer Fiesta'

(50) ;

(75) Inventor: Janet N. Egger, Wilsonville, OR (US)

(73) Assignee: Terra Nova Nurseries, Inc.

(21) Appl. No.: 12/653,640

(22) Filed: Dec. 14, 2009

(51) Int. Cl. A01H 5/00

(52) U.S. Cl. PLT/399

(57) ABSTRACT

A new and distinct form of Agastache plant characterized red flowers, large, dense flower spikes, an upright branching habit, a long bloom time, and excellent vigor.

### BOTANICAL DENOMINATION

Agastache spp.

### VARIETY DESIGNATION

‘Summer Fiesta’

### PARENTAGE

Agastache ‘Raspberry Summer’×unknown

### BACKGROUND OF THE INVENTION

The present invention relates to a new and distinct cultivar of Agastache, given the name, ‘Summer Fiesta’. Agastache is in the family Lamiaceae. This new variety is an open pollinated seedling selection out of Agastache ‘Raspberry Summer’ as such the pollen parent is unknown.

Compared to the seed parent, Agastache ‘Raspberry Summer’, the new cultivar has red rather than red purple flowers.

This plant exhibits the following characteristics that make it unique:
1. red flowers,
2. large, dense flower spikes,
3. an upright branching habit,
4. a long bloom time, and
5. excellent vigor.

The new variety has been reproduced only by asexual propagation (stem cuttings and micropropagation). Each of the progeny exhibits identical characteristics to the original plant. Asexual propagation by division and micropropagation as done in Canby, Oreg., shows that the foregoing characteristics and distinctions come true to form and are established and transmitted through succeeding propagations. The present invention has not been evaluated under all possible environmental conditions. The phenotype may vary with variations in environment without a change in the genotype of the plant.

### BRIEF DESCRIPTION OF THE DRAWINGS

FIG. 1 shows close up of a nine-month-old Agastache ‘Summer Fiesta’ growing in the field in late summer in Canby, Oreg.

FIG. 2 shows a bed of nine-month-old Agastache ‘Summer Fiesta’ growing in the field in late summer in Canby, Oreg.

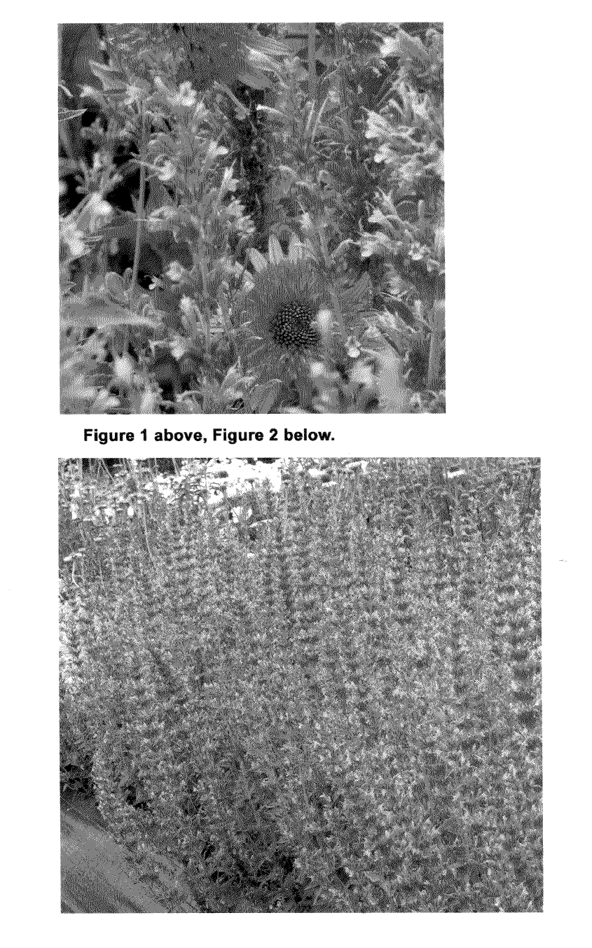

### DETAILED PLANT DESCRIPTION

The following is a detailed description of the new Agastache cultivar based on observations of eighteen-month-old specimens in August grown outside in the ground in the trial beds in Canby, Oreg. Canby is Zone 8 on the USDA Hardiness map. Temperatures range from a high of 95 degrees F. in August to an average of 32 degrees F. in January. Normal rainfall in Canby is 42.8 inches per year. The color descriptions are all based on The Royal Horticultural Society Colour Chart, 5^{th} edition.
Plant:
   Form.—upright.
   Type.—herbaceous perennial.
   Cold hardiness.—USDA Zone 7-10.
   Size.—grows to 40 cm wide and 70 cm tall to the top of the flowers.
   Vigor.—excellent.
   Branching.—about 9 main branches from the base.
   Stem.—square in cross section, grows to 7 mm wide and 30 cm tall to the first flower, sparsely pubescent, internodes range from 2 to 8 cm long, Yellow Green 146C.
   Roots.—White 155B, fibrous, plants root easily from stem tip cuttings.
Leaf:
   Type.—simple.
   Arrangement.—opposite.
   Shape.—ovate.
   Venation.—pinnate.
   Margins.—crenate to serrate.
   Apex.—acute.
   Base.—truncate to cordate.
   Blade size.—grows to 50 mm long and 30 mm wide.
   Surface texture.—very short hairs on bottom side, glabrous top side.
   Petiole description.—22 mm long and 1 mm wide, pubescent, Yellow Green 147B.
   Leaf color.—top side closest to Green N137A, bottom side closest to Yellow Green 147B.
Inflorescence:
   Type.—terminal spike with 7 to 14 whorls.
   Number of flowers.—up to 220 per spike.
   Size of inflorescence.—grows to 41 cm tall and 7 cm wide.
   Peduncle.—grows to 46 cm tall and 3 mm wide, pubescent, Yellow Green 146C.
   Pedicel.—2 to 3 mm long, pubescent, Yellow Green 146C in shade and tinted Greyed Purple 187B in sun.
   Bloom period.—July through frost in Canby, Oreg.
Flower bud:
   Size.—21 mm long and 4 mm wide at the widest point prior to opening.
   Description.—tubular.
   Surface texture.—pubescent.
   Color.—Red 51A.
Flower:
   Type.—zygomorphic.
   Shape.—bilabiate, tubular.
   Size.—30 mm long and 7 mm wide.
   Corolla description.—22 mm long and spreads to 7 mm wide; color outside Red 39A and inside Red 50A to 50B; pubescent inside and out, tube 20 mm long and 1 mm to 4 mm wide; 2 upper lobes joined 3 mm long and 2 mm wide, ovate, acute, entire; three lower lobes, two spreading sideways and curve down slightly, each 2.5 mm long and 2.5 mm wide, acute, entire; the bottom lobe broad, reflexed, obovate, notched at the tip, entire, 4 mm wide and 4 mm deep.
   Calyx description.—6 mm long and 2.5 mm wide, pubescent outside and glabrous inside, 5 lobed, lobes narrow, 2 mm long, entire, acuminate, color closest to Brown 200D.
   Pistil description.—30 mm long, ovary 0.5 mm long, Yellow Green 146C, style 19.5 mm long, White NN155D; stigma two-parted, 1 mm, Red Purple 71A
   Stamen description.—4 in number, paired and exerted, top two 10 mm long and bottom two 8 mm long, filaments Red Purple 71C, anthers 0.5 mm long, Red Purple 71C, pollen White 155A.
   Fragrance.—light herbal.
   Lastingness.—a spike blooms for about 3 weeks on the plant.
Fruit/seed: 4 nutlets, 1 mm long, ovoid, Black 202A.
Pests and diseases: Agastache are known to be susceptible to mildew and rust in dry summer months. The new cultivar has no known resistances but has shown no problems in Canby, Oreg.

I claim:

1. A new and distinct form of Agastache plant substantially as shown and described.
